(19) United States
(12) Patent Application Publication
(10) Pub. No.: US 2024/0386562 A1
(43) Pub. Date: Nov. 21, 2024

(54) AN AI BASED SYSTEM AND METHOD FOR DETECTION OF OPHTHALMIC DISEASES

(71) Applicant: Ophthalytics, Inc., Marietta, GA (US)

(72) Inventor: Awais BAJWA, Marietta, GA (US)

(73) Assignee: Ophthalytics, Inc., Marietta, GA (US)

(21) Appl. No.: 18/669,464

(22) Filed: May 20, 2024

Related U.S. Application Data
(60) Provisional application No. 63/467,927, filed on May 20, 2023.

(51) Int. Cl. G06T 7/00; A61B 3/00; A61B 3/14; G06F 21/62; G06T 7/13; G06T 7/40; G16H 15/00

(52) U.S. Cl. CPC G06T 7/0014; A61B 3/0025; A61B 3/145; G06F 21/6254; G06T 7/13; G06T 7/40; G16H 15/00; G06T 2207/10016; G06T 2207/10024; G06T 2207/20081; G06T 2207/30041; G06T 2207/30101

(57) ABSTRACT

The AI-based system for ophthalmic disease detection comprises image capturing units to record retinal videos, pre-processing modules to select and standardize retinal images, and feature extraction modules to analyze relevant features. A data analysis module compares extracted features with pre-stored images to identify potential symptoms indicative of eye diseases. An AI grading module assesses symptom severity, while a report generation module generates detailed reports, including information on macular degeneration and geographic atrophy. Through this comprehensive approach, the system offers accurate and efficient detection of ophthalmic diseases, enabling timely intervention and treatment planning.

### CROSS-REFERENCE TO RELATED APPLICATION

This application claims the benefit of priority under 35 U.S.C. § 119 (c) of U.S. Provisional Application No. 63/467,927, filed May 20, 2023, entitled “AN AI BASED SYSTEM AND METHOD FOR DETECTION OF OPTHALMIC DISEASES,” the entire content of which is hereby incorporated by reference herein in its entirety and should be considered a part of this specification.

### FIELD OF THE INVENTION

Embodiments of a present disclosure relate to diagnosis of complex ophthalmic diseases and more particularly to an Artificial Intelligence (AI) based system and method for detection of ophthalmic diseases.

### BACKGROUND OF THE INVENTION

Artificial Intelligence (AI) technology, particularly deep learning, has undergone significant advancements in recent years and has garnered increasing attention in the medical image diagnosis field. Deep learning works by utilizing multi-layered artificial neural networks that combine low-level features to create more abstract high-level features. Unlike expert systems, deep learning can better capture the fundamental characteristics of data, leading to superior results. Currently, extensive research is being conducted in the medical imaging field using deep learning, including breast cancer and lung cancer detection, cardiovascular imaging, and other pathological examinations.

Further, AI has the potential to transform the diagnosis of ophthalmic diseases by improving the efficiency and accuracy of screening and diagnosis. AI-based systems analyses vast amounts of ophthalmic data and images to detect subtle changes and abnormalities that may be missed by the human eye. In recent years, deep learning, a type of AI that uses neural networks to learn and classify data, has shown remarkable success in ophthalmic disease diagnosis.

In particular, the eye plays a crucial role in vision and is susceptible to various diseases that can cause blindness or visual impairment. While some of the diseases, like diabetes, are systemic and affect multiple organs, others, such as primary open angle glaucoma and age-related macular degeneration, are localized to the eyes. Unfortunately, there is a shortage of trained eye care providers who possess the necessary skills to diagnose both systemic diseases with ophthalmic manifestations and primarily ophthalmic diseases. This lack of expertise poses a significant burden on society, as delayed or incorrect diagnoses can lead to preventable morbidity and mortality. In light of the challenges, there has been a growing interest in the development of computer-based systems that can automate the diagnosis of ophthalmic diseases.

Further, ophthalmic diseases, such as age-related macular degeneration, Diabetic Retinopathy, and glaucoma, are leading causes of vision loss and blindness worldwide. Early detection and treatment are critical in preventing further progression of the diseases and preserving vision. However, traditional diagnostic methods, such as fundus photography and visual field testing, can be time-consuming, expensive, and require a high level of expertise.

In particular, Diabetic Retinopathy (DR) is a microvascular condition that occurs as a secondary complication to diabetes and is a leading cause of visual impairment. Timely eye examinations are crucial for early detection and treatment of DR to prevent vision loss. Currently, healthcare professionals conduct examinations. However, due to time constraints, technical challenges, and lack of equipment, patients with diabetes may find it inconvenient to attend periodic eye examinations. Furthermore, there is a significant shortage of qualified healthcare professionals who perform the examinations, particularly in rural areas. Failure to monitor the progression of DR can lead to a significant decline in visual acuity, underscoring the urgent need for accessible and reliable monitoring mechanisms.

Additionally, one of the early signs of Diabetic Retinopathy (DR) is the appearance of microaneurysms, which are small, round dilations of the retinal blood vessels. These microaneurysms can leak fluid into the surrounding tissue, causing damage to the retina and impairing vision. Detecting microaneurysms is an important diagnostic step in identifying DR, and carly detection can lead to better outcomes for patients. However, this process is often time-consuming and requires a high level of expertise. Manual screening of images by ophthalmologists is not only time-consuming but also subjective, and there can be inter-and intra-observer variability in the detection of microaneurysms. Moreover, the limited availability of qualified ophthalmologists poses a challenge for timely screening of Diabetic Retinopathy. Therefore, there is a need for an automated system that can accurately detect microaneurysms in a timely and efficient manner.

Therefore, there are a myriad of complex eye-related diseases that can result in vision loss or blindness. Timely and accurate diagnosis is essential for preserving vision, but current methods of screening and diagnosis can be time-consuming, subjective, and require a high level of expertise.

Hence, there is a need for an AI based system and method for detection of ophthalmic diseases.

### SUMMARY OF THE INVENTION

To address the foregoing problems, in whole or in part, and/or other problems that may have been observed by persons skilled in the art, the present disclosure provides compositions and methods as described by way of example as set forth below.

A principal objective of the invention is to develop an AI-based system capable of accurately detecting ophthalmic diseases by analyzing retinal images captured by image capturing units.

Another objective of the invention is to implement a pre-processing module to standardize retinal images into canonical formats, ensuring consistency and reliability in subsequent analysis.

Another objective of the invention is to create a feature extraction module to identify and extract relevant features from retinal images, such as lesions, blood vessels, and abnormalities.

Another objective of the invention is to integrate an AI grading module to analyze extracted features and assign severity levels to potential symptoms of ophthalmic diseases, facilitating precise diagnosis and treatment planning.

In view of the foregoing, the present invention provides an artificial Intelligence (AI) based system for detection of ophthalmic diseases comprises one or more image capturing units designed to record a retinal video of a patient, containing multiple retinal images from both eyes. Stored within a memory are a series of modules: a pre-processing module responsible for selecting suitable retinal images from the video and converting them into suitable image formats such as JPG, PNG, etc., a feature extraction module tasked with identifying and extracting pertinent features from the selected images, a data analysis module that evaluates or correlates these features with pre-stored images to detect potential symptoms indicative of ophthalmic diseases, an AI grading module which evaluates the severity of identified symptoms, and finally, a report generation module that produces a comprehensive report encompassing details on macular degeneration and geographic atrophy.

In an aspect of the present invention, the AI based system for detection of ophthalmic diseases includes one or more image capturing units, one or more hardware processors and a memory coupled to the one or more hardware processors and a storage module. The memory includes a plurality of modules in the form of programmable instructions executable by the one or more hardware processors. The one or more image capturing unit is configured to capture a retinal video of a patient. The retinal video is preferably 2 to 3 seconds and include of a plurality of retinal images of both left and right eyes. The image capturing unit is preferably a fundus camera. Further one or more image capturing unit is configured to receive a RGB image. The plurality of modules includes a pre-processing module configured to select one or more suitable retinal images from the fundus camera. Further the pre-processing module also transforms the one or more suitable retinal images along with the RGB image into one or more suitable image formats via conducting a set of pre-processing operations. The one or more suitable image formats are routed to a supervised machine learning model for training and testing purposes. The one or more suitable retinal images are 70% canonicalized whereas 30% processed by a different neural network and for training a set of images are captured using the one or more image capturing units of plurality of vendors. Further, a feature extraction module configured to extract features from the selected one or more retinal images. The system further includes of a data analysis module configured to match the features with a set of pre-stored images to identify one or more potential symptoms in the one or more retinal images that indicate presence of one or more ophthalmic diseases. The ophthalmic diseases refer to highly complex eye related diseases such as Diabetic Retinopathy. The one or more potential symptoms is further transferred to an AI grading module configured to analyze the one or more potential symptoms to provide a grade related to severity level. The one or more potential symptoms along with the grade is forwarded to a report generation module configured to generate a detailed report that includes macular degeneration and geographic atrophy. The generated report is further sent to an operator of the system for diagnosis or treatment plans. Additionally, the storage module configured to store the retinal video, one or more retinal images, one or more canonical image formats, one or more potential symptoms, grade, and detailed report according to a profile of the patient for record and training purpose.

In an aspect of the present invention, the image capturing unit is a fundus camera and the retinal video is preferably 2 to 3 seconds in duration.

In an aspect of the present invention, the pre-processing module further processes a RGB image received by the image capturing unit.

In an aspect of the present invention, the feature extraction module extracts features including lesions, blood vessels, microaneurysms, hemorrhages, hard-exudates, soft-exudates, venous beading, intraretinal microvascular abnormalities (IRMA), neovascularization at the disc (NVD), neovascularization of the retina elsewhere (NVE), fovea, optic disc, laser mark, and abnormal blood vessel growth.

In an aspect of the present invention, the AI grading module provides grades including no apparent retinopathy, mild non-proliferative diabetic retinopathy, moderate non-proliferative diabetic retinopathy, severe non-proliferative diabetic retinopathy, and proliferative diabetic retinopathy.

In an aspect of the present invention, the storage module stores the retinal video, retinal images, canonical image formats, potential symptoms, grade, and detailed report according to a profile of the patient.

In an aspect of the present invention, the system employs deep learning algorithms for analysis of retinal images and videos.

In accordance with another embodiment of the present disclosure, the present invention discloses a method for AI-based detection of ophthalmic diseases using an AI-based system encompasses several steps. Firstly, one or more retinal images of a patient's eye are captured utilizing one or more image capturing units, comprising a variety of images from both left and right eyes. Subsequently, the captured retinal images undergo pre-processing to convert them into one or more canonical image formats. Following this, features are extracted from the pre-processed retinal images. These features are then analyzed to identify potential symptoms indicative of various eye diseases. Upon identification, the potential symptoms are graded to ascertain the severity level of the detected eye diseases. Finally, a detailed report is generated based on the analysis and grading of the retinal images, encompassing information regarding macular degeneration and geographic atrophy, providing comprehensive insights into the patient's eye health condition.

In an aspect of the present invention, the AI based method for detection of ophthalmic diseases includes capturing one or more images of eye of a patient by a fundus camera. The one or more images are analyzed by utilizing a plurality of artificial intelligence techniques to identify one or more potential symptoms that indicate presence of one or more eye diseases. The ophthalmic diseases refer to highly complex eye related diseases such as Diabetic Retinopathy. Further, the method includes generating a detailed report within 3 to 6 seconds which is further assessed by a medical practitioner like physician or ophthalmologist for a treatment process. The generated report provides a data regarding the stage or level of severity of the one or more eye diseases which assist the medical practitioner in determination of a suitable treatment plan. The stage or level of severity includes severe, mild, moderate, and proliferative.

In accordance with another embodiment of the present disclosure, an AI based method for detecting and grading of ophthalmic diseases is disclosed. The AI based method for detecting and grading of ophthalmic diseases includes capturing one or more retinal images of an eye of a patient. The one or more retinal images are analyzed by utilizing a plurality of artificial intelligence techniques to identify one or more potential symptoms that indicate presence of one or more eye diseases. The ophthalmic diseases refer to highly complex eye related diseases such as Diabetic Retinopathy. Further the method includes generating a detailed report within 3 to 6 seconds which is further assessed by a medical practitioner like physician or ophthalmologist for a treatment process. The generated report provides a data regarding the stage or level of severity of the one or more eye diseases and lesion localization which assist the medical practitioner in determination of a suitable treatment plan. The stage or level of severity includes severe, mild, moderate, and proliferative. The method allows for fast and efficient screenings for signs of Diabetic Retinopathy (DR) and age-related macular degeneration (AMD) using fundus images. Further the method provides the stage or level of severity which allows for more accurate and effective diagnosis.

In aspect of the present invention, the pre-processing step includes adjusting resolution, color space, and aspect ratio of the retinal images to create consistent baseline for analysis.

In aspect of the present invention, the storing step further includes organizing the stored data according to specific characteristics of the patient for efficient retrieval and analysis.

In aspect of the present invention, the pre-processing step includes adjusting resolution, color space, and aspect ratio of the retinal images.

In aspect of the present invention, the analyzing step further comprises comparing the extracted features with a set of pre-stored images to identify patterns indicative of specific ophthalmic diseases.

In aspect of the present invention, the storing step further includes anonymizing the stored data to ensure patient privacy.

In aspect of the present invention, the pre-processing step includes employing edge detection algorithms to identify boundaries of blood vessels or lesions, and texture analysis techniques to detect abnormal patterns or colors in the retinal images.

Additional features of the invention will be or will become apparent to one with skill in the art upon examination of the following figures and detailed description. It is intended that all such additional features and advantages be included within this description, be within the scope of the invention, and be protected by the accompanying claims.

### BRIEF DESCRIPTION OF THE DRAWINGS

Having thus described the subject matter of the present invention in general terms, reference will now be made to the accompanying drawings, which are not necessarily drawn to scale, and wherein:

FIG. 1 is a block diagram illustrating an exemplary Artificial Intelligence (AI) based system for detection of ophthalmic diseases is disclosed, in accordance with an embodiment of the present disclosure;

FIG. 2 is another block diagram illustrating an exemplary AI based system for detection of ophthalmic diseases is disclosed, in accordance with an embodiment of the present disclosure;

FIG. 3 is block diagram illustrating an working of an exemplary AI based system for detection of ophthalmic diseases with the storage module is disclosed, in accordance with an embodiment of the present disclosure;

FIG. 4 is another block diagram illustrating a working of an exemplary AI based system for detection of ophthalmic diseases is disclosed, in accordance with an embodiment of the present disclosure.

FIG. 5 Shows a process flow chart of the Artificial Intelligence (AI) based system for detection of ophthalmic diseases, in accordance with an embodiment of the present disclosure.

Skilled artisans will appreciate that elements in the drawings are illustrated for simplicity and may not have necessarily been drawn to scale. Furthermore, in terms of the construction of the device, one or more components of the device may have been represented in the drawings by conventional symbols, and the drawings may show only those specific details that are pertinent to understanding the embodiments of the present invention so as not to obscure the drawings with details that will be readily apparent to those of ordinary skill in the art having the benefit of the description herein.

### DETAILED DESCRIPTION OF THE INVENTION

The subject matter of the present invention now will be described more fully hereinafter with reference to the accompanying drawings, in which some, but not all embodiments of the subject matter of the present invention are shown. Like numbers refer to like elements throughout. The subject matter of the present invention may be embodied in many different forms and should not be construed as limited to the embodiments set forth herein; rather, these embodiments are provided so that this disclosure will satisfy applicable legal requirements. Indeed, many modifications and other embodiments of the subject matter of the present invention set forth herein will come to mind to one skilled in the art to which the subject matter of the present invention pertains having the benefit of the teachings presented in the foregoing descriptions and the associated drawings. All illustrations of the drawings are for the purpose of describing selected versions of the present invention and are not intended to limit the scope of the present invention. Therefore, it is to be understood that the subject matter of the present invention is not to be limited to the specific embodiments disclosed and that modifications and other embodiments are intended to be included within the scope of the appended claims.

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and example of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein-as understood by the ordinary artisan based on the contextual use of such term-differs in any way from any particular dictionary definition of such term, it is intended that the meaning of the term as understood by the ordinary artisan should prevail.

Furthermore, it is important to note that, as used herein, “a” and “an” each generally denotes “at least one”, but does not exclude a plurality unless the contextual use dictates otherwise. When used herein to join a list of items, “or” denotes “at least one of the items”, but does not exclude a plurality of items of the list. Finally, when used herein to join a list of items, “and” denotes “all of the items of the list”.

The present invention introduces an innovative Artificial Intelligence (AI) based system and method for the detection of ophthalmic diseases, revolutionizing the way healthcare professionals diagnose and treat eye conditions. The invention discloses an AI system comprising multiple image capturing units, memory modules housing various processing components, and specialized algorithms designed to analyze retinal images with unparalleled precision. This system is capable of capturing retinal videos, extracting essential features, and generating detailed reports, all aimed at facilitating carly diagnosis and treatment of complex eye diseases.

The pre-processing module disclosed in the invention selects suitable retinal images from videos and transform them into canonical formats, ensuring consistency and reliability in subsequent analysis. Subsequently, the feature extraction module identifies key features within these images, crucial for identifying potential symptoms indicative of various eye diseases. These features are then meticulously analyzed by the data analysis module, which leverages pre-stored images to detect and diagnose ophthalmic diseases with remarkable accuracy.

Moreover, the invention includes an AI grading module, which plays a pivotal role in determining the severity levels of identified symptoms, providing clinicians with valuable insights for treatment planning. The generated reports, inclusive of details on macular degeneration and geographic atrophy, serve as comprehensive summaries of the patient's eye health, aiding healthcare professionals in making informed decisions.

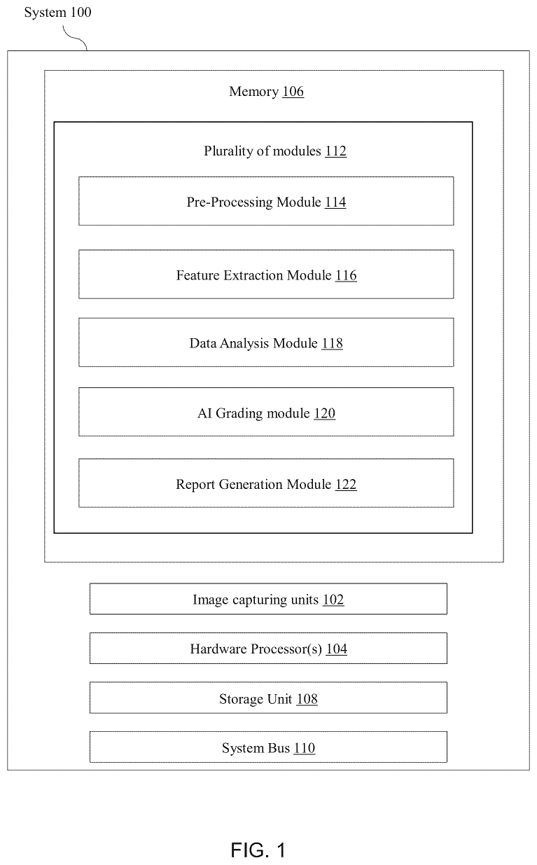

Referring now to the drawings, and more particularly to FIG. 1 through FIG. 4, where similar reference characters denote corresponding features consistently throughout the figures, there are shown preferred embodiments and these embodiments are described in the context of the following exemplary system and/or method.

FIG. 1 is a block diagram illustrating an exemplary Artificial Intelligence (AI) based system for detection of ophthalmic diseases is disclosed, in accordance with an embodiment of the present disclosure. According to FIG. 1, the AI based system 100 for detection of ophthalmic diseases includes one or more image capturing units 102, one or more hardware processors 104, a memory 106, a storage unit 108, a system bus 110 and a plurality of modules 112. The one or more image capturing unit 102 is configured to capture a retinal video of a patient, where the retinal video is preferably 2 to 3 seconds and includes a plurality of retinal images of both left and right eyes. The image capturing unit 102 is preferably a fundus camera. The AI based system for detection of ophthalmic diseases receives two types of input images, a RGB image and the retinal video or retinal image. The one or more hardware processors 104, the memory 106, the storage unit 108 are communicatively coupled through the system bus 110 or any similar mechanism. In an embodiment of the present disclosure, memory 106 is coupled to one or more hardware processors 104. Memory 106 includes the plurality of modules 112 in the form of programmable instructions executable by one or more hardware processors 104. In an embodiment of the present disclosure the plurality of modules 112 includes a pre-processing module 114, a feature extraction module 116, a data analysis module 118, an AI grading module 120, and a report generation module 122. Details on the plurality of modules 112 have been elaborated in subsequent paragraphs of the present description.

The one or more hardware processors 104, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 104 may also include embedded controllers, such as generic or programmable logic devices or arrays, application specific integrated circuits, single-chip computers, and the like. The storage unit 108 may be a cloud storage or a local file directory within a remote server.

The memory 106 may be non-transitory volatile memory and non-volatile memory. The memory 106 may be coupled for communication with the one or more hardware processors 104, such as being a computer-readable storage medium. The one or more hardware processors 104 may execute machine-readable instructions and/or source code stored in the memory 106. A variety of machine-readable instructions may be stored in and accessed from the memory 106. The memory 106 may include any suitable elements for storing data and machine-readable instructions, such as read only memory, random access memory, erasable programmable read only memory, electrically crasable programmable read only memory, a hard drive, a removable media drive for handling compact disks, digital video disks, diskettes, magnetic tape cartridges, memory cards, and the like. In the present embodiment, the memory 106 includes the plurality of modules 112 stored in the form of machine-readable instructions on any of the above-mentioned storage media and may be in communication with and executed by the one or more hardware processors 104.

The pre-processing module 114 is configured to select one or more suitable retinal images from the fundus camera. In an embodiment of the present disclosure, the pre-processing module 114 also transforms the one or more suitable retinal images into one or more canonical image formats via conducting a set of pre-processing operations. Further, a RGB image is processed by the pre-processing module 114 that canonicalizes the image and later the image is sent to multiple groups for processing on the basis of camera/device features. The retinal video is converted into video clip that is converted to images, because I second of video click has 24 frames per second, all these images are evaluated in the preprocessing pipeline and the pre-processing module 114 select a top image with the best output for conversion into the canonical image. The canonical image format used herein refers to a standardized image format that has been defined to ensure consistency in the way images are processed and analyzed by the system and to make the one or more suitable retinal images more appropriate for analysis and comparison. The transformation of the one or more suitable retinal images into the one or more canonical image formats involves adjustment in resolution, color space, aspect ratio and alike properties to create a consistent baseline for the analysis, resulting in casier comparison, improvement in accuracy, reliability of the analysis, particularly in cases where the one or more retinal images may have been captured under different conditions or using different equipment.

The feature extraction module 116 is configured to extract features from the selected one or more retinal images. In an exemplary embodiment of the present disclosure, the one or more features include but not limited to presence of lesions, blood vessels, or other abnormalities like microaneurysms, hemorrhages, hard-exudates, soft-exudates, venous beading, intraretinal microvascular abnormalities (IRMA), neovascularization at the disc (NVD), neovascularization of the retina elsewhere (NVE), fovea, optic disc, laser mark, and abnormal blood vessel growth. Furthermore, the features include relevant information or characteristics from retinal images that are used to identify signs of disease. There are several types of features that are extracted from retinal images, depending on the specific disease being targeted and the imaging modality used. For example, in Diabetic Retinopathy, which is a common cause of blindness in diabetic patients, key features that can be extracted from retinal images include microaneurysms, hemorrhages, hard-exudates, soft-exudates, venous beading, intraretinal microvascular abnormalities (IRMA), neovascularization at the disc (NVD), neovascularization of the retina elsewhere (NVE), fovea, optic disc, laser mark, and abnormal blood vessel growth. Moreover, to extract the features, the feature extraction module 116 may use a combination of image processing techniques and machine learning algorithms. For instance, the system 100 may use edge detection algorithms to identify the boundaries of blood vessels or lesions or use texture analysis techniques to identify regions of the image with abnormal patterns or colors. The features are used by a data analysis module 118 learn to recognize patterns in the data and make a diagnosis based on the presence or absence of specific features.

The data analysis module 118 is configured to match features with a set of pre-stored images to identify one or more potential symptoms in the one or more retinal images that indicate presence of one or more eye diseases. Eye diseases refers to highly complex eye related diseases such as Diabetic Retinopathy. The data analysis module 118 is able to target the highly complex eye disease which is difficult to diagnose without the use of advanced imaging and analysis techniques. For example, Diabetic Retinopathy causes damage to the blood vessels in the retina, leading to vision loss if left untreated. The data analysis module 118 identifies the features associated with the complex eye diseases, so that the medical practitioners or clinicians obtain more accurate and reliable diagnoses, which can lead to carlier intervention and better outcomes for patients.

The one or more potential symptoms is further transferred to the Artificial Intelligence (AI) grading module 120 configured to analyze the one or more potential symptoms to provide a grade related to severity level. In an exemplary embodiment the grade include no apparent retinopathy (grade 0), mild Non-proliferative Diabetic Retinopathy (grade 1), moderate Non-proliferative Diabetic Retinopathy (grade 2), severe Non-proliferative Diabetic Retinopathy (grade 3), and proliferative Diabetic Retinopathy (grade 4). The AI grading module 120 is programmed to analyze the one or more potential symptoms and provide a grade related to the severity level of the detected symptoms. The grade helps medical practitioners or clinicians to determine the appropriate course of treatment for the patient and monitor the condition over time. The AI grading module 120 uses advanced algorithms and machine learning techniques to analyze the one or more potential symptoms and assign a grade based on the severity of the symptoms detected. The severity level of the symptoms may be determined by a range of factors, including the type of eye disease, the extent of the damage or abnormality detected, and the patient's overall health status. The AI grading module 120 may also provide additional information, such as the stage of the disease, the likelihood of progression, and recommended treatment options. By using an AI grading module 120 to analyze the potential symptoms of eye diseases, the medical practitioners or clinicians can obtain more accurate and reliable information about the patient's condition, which can lead to better outcomes and improved quality of care.

The one or more potential symptoms along with the grade is forwarded to a report generation module 122 configured to generate a detailed report that includes macular degeneration and geographic atrophy. The generated report is further sent to an operator of the system for diagnosis or treatment plans. The report generation module 122 is specifically programmed to generate the detailed report that includes the potential presence of macular degeneration and geographic atrophy, as well as other relevant information about the patient's condition. The detailed report may include images of the patient's retina, descriptions of the potential symptoms detected, and recommendations for further testing or treatment. The detailed report is then sent to an operator of the system, who may be a healthcare professional such as an ophthalmologist, optometrist, or technician. The operator uses the report to diagnose the patient's condition and develop an appropriate treatment plan. The detailed report is shared with other healthcare providers involved in the patient's care to ensure that all relevant information is available to inform treatment decisions.

The data analysis module 118, the AI grading module 120, and the report generation module 122 utilizes deep learning algorithms to analyze the images and videos of the patient's eyes to detect any signs of pathology. In the case of video-based reports, the algorithm analyzes all the images in the video and selects the best two images for the right and left eyes for the final diagnosis report. The system 100 also employs an AI-based localization algorithm to identify the location of the pathology for geographic atrophy and generates a trend analysis report, which allows physicians and ophthalmologists to better understand the progression of the disease and create a more effective treatment plan. The deep learning algorithms are trained on large datasets of retinal images and videos to improve the accuracy of the diagnosis and reduce the risk of false positives or false negatives.

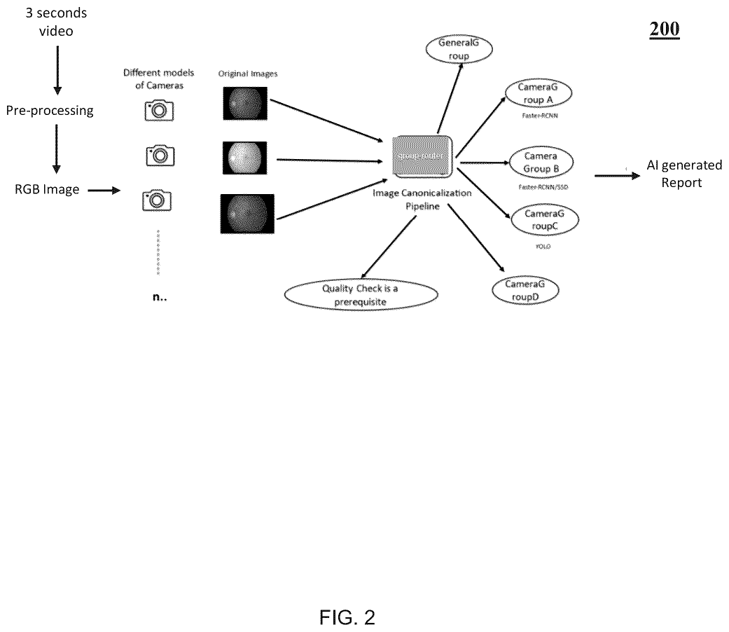

FIG. 2 is another block diagram illustrating an exemplary AI based system for detection of ophthalmic diseases is disclosed, in accordance with an embodiment of the present disclosure. Further, in the system 200, one or more canonical image formats are routed to a supervised machine learning model for training and testing purposes. In an embodiment of the present disclosure the one or more suitable retinal images are 70% canonicalized whereas 30% processed by a different neural network and for training a set of images are captured using the one or more image capturing units of plurality of vendors. In an exemplary embodiment of the present disclosure, the neural networks include but not limited to Faster R-CNN, YOLOv3 & YOLOv4, Single Shot MultiBox Detector, RetinaNet. Mask R-CNN and CenterNet.

The one or more canonical image formats are routed to different neural networks based on the similarities in quality and output. Further a router is used to route the one or more canonical image formats to the corresponding CameraGroup, which is a group of images that share similar quality and output characteristics. The groups are supervised learning blocks that have been trained on datasets from the one or more image capturing units.

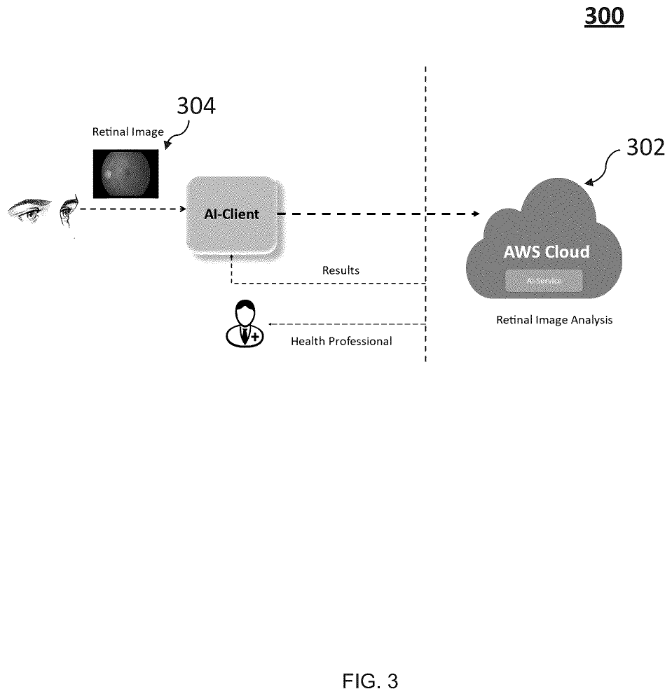

FIG. 3 is block diagram illustrating a working of an exemplary AI based system for detection of ophthalmic diseases with the storage module is disclosed, in accordance with an embodiment of the present disclosure. Additionally, the system 300 includes a storage module 302 configured to store the retinal video, one or more retinal images 304, one or more canonical image formats, one or more potential symptoms, grade and detailed report according to a profile of the patient for record and training purpose of the system 300. The storage module 302 is configured to store various types of data related to the patient's eye health which includes the retinal video, one or more retinal images 304, and one or more canonical image formats that are generated by the system 300 during the screening process. In addition to storing the images 304, the storage module 302 also stores information related to the potential symptoms that are detected, as well as the grade assigned by the AI grading module 120 (as depicted in FIG. 1) which is further used to generate the detailed report and is also stored for record-keeping purposes. The storage module 302 is programmed to store this data according to a profile of the patient, which may include information such as the patient's name, age, medical history, and any other relevant information which allows the system to easily access and retrieve data for future reference and for training purposes.

The system offers several benefits to users, including speed and accuracy, a user-friendly interface, access to patient data, and cost savings. The system is constructed to provide fast and accurate assessment, enabling users to quickly evaluate patient data with ease. Additionally, the system provides access to existing patient assessments, allowing users to retrieve and review patient data anytime. The system also saves significant costs on infrastructure upgrades to serve more patients and improves return on investment. With the benefits, the system is an efficient and effective tool for assessing and managing eye diseases.

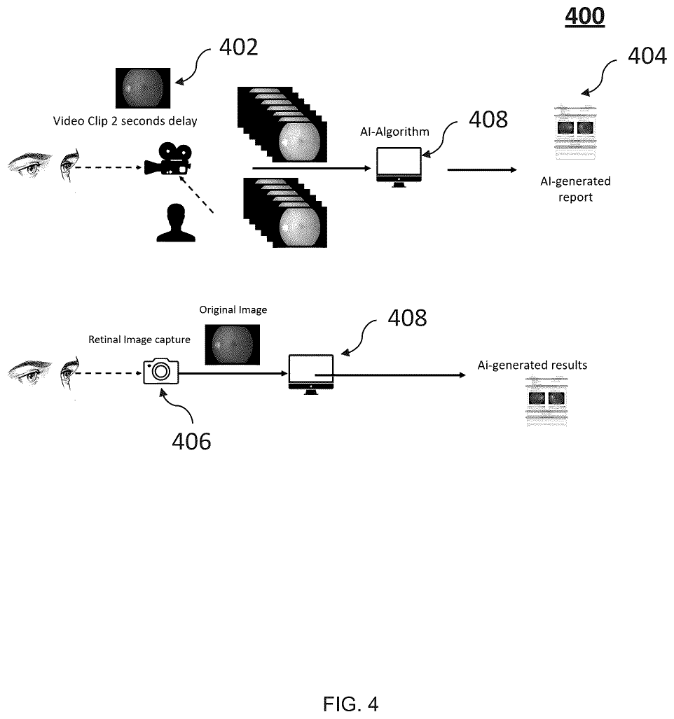

FIG. 4 is another block diagram illustrating the working of an exemplary AI based system for detection of ophthalmic diseases is disclosed, in accordance with an embodiment of the present disclosure. The system 400 for AI based detection of ophthalmic diseases provides a fast and efficient screening process for Diabetic Retinopathy (DR) detection in both clinical and retail settings. The system operates in three phases, in first phase the patient's retinal images or video 402 being captured and processed in second phase and a detailed report 404 generated within 3 to 6 seconds in third phase. The detailed report 404 includes information on the stage and severity of the disease, enabling patients to take appropriate actions and seck the right treatment. Further the system 400 includes real-time image quality check which ensures that the submitted photos are of sufficient quality for accurate reading. The system 400 can be integrated into clinical workflows, offering automated screening by analyzing a patient's retinal images taken using a fundus camera 406. The system 400 is primarily cloud-based and can be installed on a Windows PC with internet access, making an accessible and cost-efficient solution. Furthermore, the AI based system 400 for detection of ophthalmic diseases offers several key benefits, including cost-efficiency and time saving by avoiding expensive infrastructure upgrades, providing fast and secure results, and having an easy-to-use interface 408 that simplifies the evaluation of patient data. The integration of AI based system 400 for detection of ophthalmic diseases with existing systems requires no additional software, allowing for a seamless and efficient screening process. Overall, the system 400 for Artificial Intelligence (AI) based detection of ophthalmic diseases provides a reliable and accessible solution for the early detection and treatment of diseases, improving patient outcomes and quality of life.

In an embodiment of the present disclosure, an AI based method for detection of ophthalmic diseases is disclosed. The AI based method for detection of ophthalmic diseases includes capturing of one or more images of eye of a patient by a fundus camera. The one or more images are analyzed by utilizing a plurality of artificial intelligence techniques to identify one or more potential symptoms that indicate presence of one or more eye diseases. Ophthalmic diseases refer to highly complex eye related diseases such as Diabetic Retinopathy. Further the method includes generating a detailed report within 3 to 6 seconds which is further assessed by a medical practitioner like physician or ophthalmologist for a treatment process. The generated report provides a data regarding the stage or level of severity of the one or more eye diseases which assist the medical practitioner in determination of a suitable treatment plan. The stage or level of severity includes severe, mild, moderate, and proliferative.

In an embodiment of the present disclosure, another AI based method for detecting and grading of ophthalmic diseases is disclosed. The AI based method for detecting and grading of ophthalmic diseases includes capturing one or more retinal images of an eye of a patient. The one or more retinal images are analyzed by utilizing a plurality of artificial intelligence techniques to identify one or more potential symptoms that indicate presence of one or more eye diseases. Ophthalmic diseases refer to highly complex eye related diseases such as Diabetic Retinopathy. Further the method includes generating a detailed report within 3 to 6 seconds which is further assessed by a medical practitioner like physician or ophthalmologist for a treatment process. The generated report provides a data regarding the stage or level of severity of the one or more eye diseases and lesion localization which assist the medical practitioner in determination of a suitable treatment plan. The stage or level of severity includes severe, mild, moderate, and proliferative. The method allows for fast and efficient screenings for signs of Diabetic Retinopathy (DR) and age-related macular degeneration (AMD) using fundus images. Further the method provides the stage or level of severity which allows for more accurate and effective diagnosis.

The AI based system and method for detection of ophthalmic diseases is revolutionizing the way hospitals and clinics perform screenings for eye diseases. With the ability to reduce up to 60% of the workload, is a cost-efficient and time-saving solution. The AI based system and method for detection of ophthalmic diseases is device-agnostic and works with any fundus camera and performs real-time quality assessment to ensure that the submitted photos can be read. The role of screening is critical in analyzing and identifying various patterns of lesions based on thousands of images. The AI based system and method for detection of ophthalmic diseases works by extensively analyzing retinal images to identify any diseases that are seen in the eye's nerve endings and is achieved by capturing images of the patient's retina with a camera adapter and uploading them to the software to screen for any abnormalities. Upon completing the screening, the AI based system and method for detection of ophthalmic diseases generates a detailed report that is sent directly to the physician for review. The detailed reports give the physician a comprehensive summary of any identified issues, allowing them to develop a treatment plan immediately which saves time for both the physician and the patient, and ensures that treatment can begin as early as possible, leading to better outcomes.

In accordance with an embodiment, the invention lies in its device-agnostic approach to diagnosing ophthalmic diseases using artificial intelligence (AI). This device-agnostic capability, also referred to as camera-independent or device-independent functionality, allows the system to process images captured from a variety of imaging devices seamlessly. The core concept involves standardizing and canonicalizing images, ensuring that they meet a uniform format regardless of the capturing device. This standardization is crucial for maintaining consistency and accuracy in the analysis, as it eliminates variations introduced by different cameras or imaging devices.

Once the images are standardized, they are routed into multiple groups using a neural network-based router. This router intelligently directs the images to device-specific processing groups, each comprising specialized neural networks. These groups, referred to as Group A, Group B, and so forth, are designed to handle images from specific types of cameras or devices. Each group's neural network has been trained extensively on images captured by its corresponding device type, ensuring optimal performance and accuracy in recognizing ophthalmic disease indicators. This group-based neural network architecture enables the system to leverage the strengths and compensate for the weaknesses of various imaging devices.

The specialized neural networks within each group are fine-tuned to process device-specific images with high precision. By being trained on images from relevant cameras, these networks can account for unique characteristics and potential distortions inherent to those devices. This tailored processing enhances the system's ability to detect and visualize anatomical structures and potential indicators of ophthalmic diseases accurately. Consequently, the device-agnostic nature of the system not only broadens its applicability across different imaging hardware but also ensures that the diagnostic outputs are reliable and consistent, irrespective of the device used for image capture.

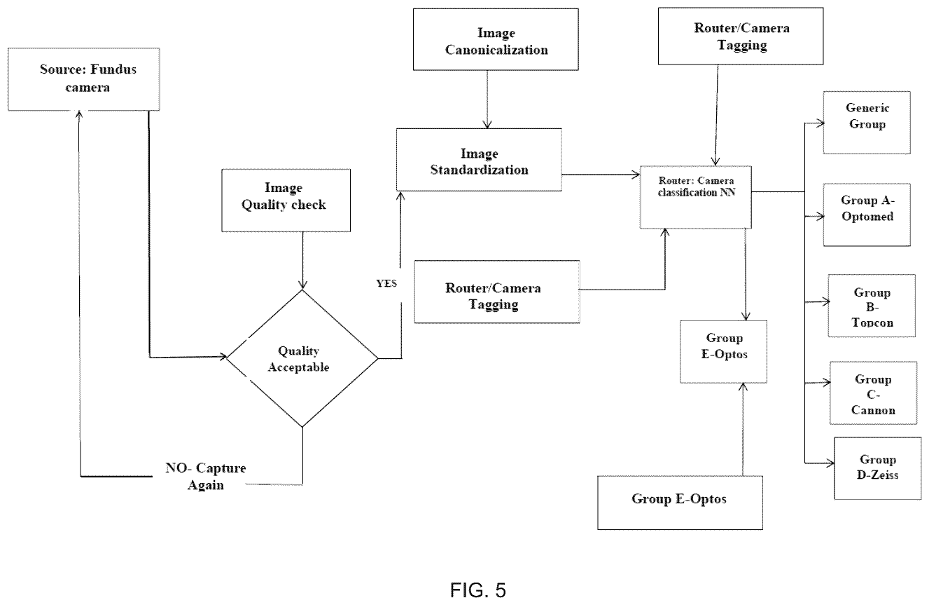

In accordance with an embodiment of the present invention, FIG. 5 Shows a process flow chart of the Artificial Intelligence (AI) based system for detection of ophthalmic diseases. The flow chart of the invention as shown begins with the capture of retinal images or videos using one or more image capturing units. These units are configured to capture high-resolution images of both the left and right eyes of a patient. Once the retinal images are acquired, they undergo an initial quality check to ensure that the images meet the required standards for further analysis. This quality check filters out images that are blurry, poorly illuminated, or otherwise unsuitable for diagnostic purposes. Next, the images proceed to the pre-processing stage, where they are transformed into canonical image formats. This involves standardizing the images to ensure uniformity in size, orientation, and resolution, making them suitable for consistent analysis. During this stage, images may also be tagged with relevant metadata, such as patient information and capture conditions, using a router/camera tagging system. This tagging helps in organizing and retrieving images efficiently for subsequent steps.

After standardization and tagging, the images are processed through a feature extraction module, which identifies and extracts critical features from the retinal images. These features include anatomical structures and potential indicators of ophthalmic diseases. The extracted features are then analyzed by a data analysis module, which compares them with pre-stored reference images to identify any potential symptoms of ophthalmic conditions. Based on this analysis, an AI grading module evaluates the severity of the identified symptoms, providing a detailed grading of the disease's progression. Finally, a report generation module compiles the findings into a comprehensive report, including diagnoses of conditions such as macular degeneration and geographic atrophy, which can be used by healthcare professionals for further patient management and treatment planning.

The one or more hardware processors, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors may also include embedded controllers, such as generic or programmable logic devices or arrays, application specific integrated circuits, single-chip computers, and the like.

The memory may be non-transitory volatile memory and non-volatile memory. The memory may be coupled for communication with the one or more hardware processors, such as being a computer-readable storage medium. The one or more hardware processors may execute machine-readable instructions and/or source code stored in the memory 106. A variety of machine-readable instructions may be stored in and accessed from the memory. The memory may include any suitable elements for storing data and machine-readable instructions, such as read only memory, random access memory, erasable programmable read only memory, electrically erasable programmable read only memory, a hard drive, a removable media drive for handling compact disks, digital video disks, diskettes, magnetic tape cartridges, memory cards, and the like. In the present embodiment, the memory includes the plurality of modules stored in the form of machine-readable instructions on any of the above-mentioned storage media and may be in communication with and executed by the one or more hardware processors. The storage unit may be a cloud storage or a local file directory within a remote server.

The non-transitory computer-readable storage medium may be any combination of one or more computer-readable media. The computer-readable medium may be a computer-readable signal medium or a computer-readable storage medium. The computer-readable storage medium may be, but is not limited to, for example, an electrical, magnetic, optical, electromagnetic, infrared or semiconductor system, device or apparatus or any combination thereof. More specific examples (non-exhaustive list) of the computer-readable storage medium include an electrical connector with one or more wires, a portable computer disk, a hard disk, a RAM, a ROM, an Erasable Programmable ROM (EPROM) or a flash memory, an optical fiber, a portable CD-ROM, an optical storage device, a magnetic storage device, or any proper combination thereof. In the application, the computer-readable storage medium may be any tangible medium including or storing a program that may be used by or in combination with an instruction execution system, device, or apparatus.

The method may be implemented in any suitable hardware, software, firmware, or combination thereof.

Thus, various embodiments of the present invention provide a solution for ophthalmic diseases, specifically focusing on the early detection of Diabetic Retinopathy (DR), as the Diabetic Retinopathy is a leading cause of blindness, and the existing diagnostic process is challenging, time-consuming, and requires skilled personnel. The AI based system and method for detection of ophthalmic diseases offer autonomous detection of Diabetic Retinopathy and age-related macular degeneration, respectively, using AI algorithms. The AI based system and method for detection of ophthalmic diseases provides cost-efficient, time-saving, and provide real-time detailed reports that is used by ophthalmologists to determine the right plan of action and level of urgency by early detection of eye diseases which also enables patients to receive timely and appropriate care. The AI based system and method for detection of ophthalmic diseases prevents permanent vision loss and improves the quality of life.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar clements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/computer system in accordance with the embodiments herein. The system herein comprises at least one processor or central processing unit (CPU). The CPUs are interconnected via system bus 110 to various devices such as a random-access memory (RAM), read-only memory (ROM), and an input/output (I/O) adapter. The I/O adapter can connect to peripheral devices, such as disk units and tape drives, or other program storage devices that are readable by the system. The system can read the inventive instructions on the program storage devices and follow these instructions to execute the methodology of the embodiments herein.

The system further includes a user interface adapter that connects a keyboard, mouse, speaker, microphone, and/or other user interface devices such as a touch screen device (not shown) to the bus to gather user input. Additionally, a communication adapter connects the bus to a data processing network, and a display adapter connects the bus to a display device which may be embodied as an output device such as a monitor, printer, or transmitter, for example.

Some of the non-limiting advantages of the present invention are:
Accurate Detection: The AI-based system enables precise identification of ophthalmic diseases by analyzing retinal images with high accuracy and reliability.
Timely Intervention: By providing fast and efficient screenings, the invention facilitates early detection of eye diseases, allowing for timely medical intervention and treatment.
Comprehensive Reports: Detailed reports generated by the system offer comprehensive insights into the patient's eye health, including information on macular degeneration and geographic atrophy, aiding clinicians in diagnosis and treatment planning.
Standardized Analysis: The pre-processing module ensures standardization of retinal images into canonical formats, enhancing consistency and reliability in subsequent analysis.
Cost Efficiency: The system's efficiency in disease detection and diagnosis helps reduce healthcare costs by streamlining processes and minimizing the need for extensive manual intervention.

Terms and phrases used in this document, and variations thereof, unless otherwise expressly stated, should be construed as open-ended as opposed to limiting. As examples of the foregoing: the term “including” should be read as mean “including, without limitation” or the like; the term “example” is used to provide exemplary instances of the item in the discussion, not an exhaustive or limiting list thereof; and adjectives such as “conventional,” “traditional,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although item, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the subject matter of the present invention. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. Although the foregoing subject matter has been described in some detail by way of illustration and example for purposes of clarity of understanding, it will be understood by those skilled in the art that certain changes and modifications can be practiced within the scope of the appended claims.

What is claimed is:

1. An Artificial Intelligence (AI) based system for detection of ophthalmic diseases, comprising:
one or more image capturing units for capturing a retinal video of a patient, wherein the retinal video comprises a plurality of retinal images of both left and right eyes;
a plurality of modules stored in a memory, comprising:
a pre-processing module configured to select one or more suitable retinal images from the retinal video and transform the retinal images into canonical image formats;
a feature extraction module configured to extract one or more features from the selected retinal images;
a data analysis module configured to match the features with pre-stored images to identify potential symptoms indicating presence of ophthalmic diseases;
an AI grading module configured to analyze the potential symptoms and provide a grade related to severity level; and
a report generation module configured to generate a detailed report including macular degeneration and geographic atrophy.

2. The system of claim 1, wherein the image capturing unit is a fundus camera.

3. The system of claim 1, wherein the retinal video is preferably 2 to 3 seconds in duration.

4. The system of claim 1, wherein the pre-processing module further processes a RGB image received by the image capturing unit.

5. The system of claim 1, wherein the feature extraction module extracts features including lesions, blood vessels, microaneurysms, hemorrhages, hard-exudates, soft-exudates, venous beading, intraretinal microvascular abnormalities (IRMA), neovascularization at the disc (NVD), neovascularization of the retina elsewhere (NVE), fovea, optic disc, laser mark, and abnormal blood vessel growth.

6. The system of claim 1, wherein the AI grading module provides grades including no apparent retinopathy, mild non-proliferative diabetic retinopathy, moderate non-proliferative diabetic retinopathy, severe non-proliferative diabetic retinopathy, and proliferative diabetic retinopathy.

7. The system of claim 1, wherein the storage module stores the retinal video, retinal images, canonical image formats, potential symptoms, grade, and detailed report according to a profile of the patient.

8. The system of claim 1, wherein the system employs deep learning algorithms for analysis of retinal images and videos.

9. A method for AI-based detection of ophthalmic diseases using an AI-based system, the method comprising:
capturing one or more retinal images of an eye of a patient using one or more image capturing units, wherein the retinal images include a plurality of retinal images of both left and right eyes;
pre-processing the retinal images to transform them into one or more canonical image formats;
extracting one or more features from the pre-processed retinal images;
analyzing the features to identify potential symptoms indicative of one or more eye diseases;
grading the potential symptoms to determine a severity level of the identified eye diseases; and
generating a detailed report based on the analysis and grading of the retinal images, wherein the detailed report includes macular degeneration and geographic atrophy.

10. The method of claim 9, wherein the pre-processing step includes adjusting resolution, color space, and aspect ratio of the retinal images to create consistent baseline for analysis.

11. The method of claim 9, wherein the storing step further includes organizing the stored data according to specific characteristics of the patient for efficient retrieval and analysis.

12. The method of claim 9, wherein the pre-processing step includes adjusting resolution, color space, and aspect ratio of the retinal images.

13. The method of claim 9, wherein the analyzing step further comprises comparing the extracted features with a set of pre-stored images to identify patterns indicative of specific ophthalmic diseases.

14. The method of claim 9, wherein the storing step further includes anonymizing the stored data to ensure patient privacy.

15. The method of claim 9, wherein the pre-processing step includes employing edge detection algorithms to identify boundaries of blood vessels or lesions, and texture analysis techniques to detect abnormal patterns or colors in the retinal images.
